(19) United States
(12) Patent Application Publication
(10) Pub. No.: US 2016/0046694 A1
(43) Pub. Date: Feb. 18, 2016

(54) METHODS AND CONSTRUCTS FOR EXPRESSING BIOLOGICALLY ACTIVE PROTEINS IN MAMMALIAN CELLS

(71) Applicant: USHA BIOTECH LIMITED, Vengal Rao Nagar (IN)

(72) Inventors: Raj Kumar KUNAPARAJU, Vengal Rao Nagar (IN); Bindu KODATI, Vengal Rao Nagar (IN); Radhika NADIMPALLI, Vengal Rao Nagar (IN)

(21) Appl. No.: 14/780,592

(22) PCT Filed: Mar. 28, 2014
(86) PCT No.: PCT/IN2014/000198

§ 371 Date: Sep. 28, 2015

(30) Foreign Application Priority Data
Mar. 30, 2013 (IN) 1443/CHE/2013

(51) Int. Cl. C07K 16/00

(52) U.S. Cl. CPC C07K 16/00; C07K 2317/14

(57) ABSTRACT

Methods and constructs for expressing biologically active proteins in eukaryotic cells are disclosed. A method for producing a non-conventional expression vector for production of biologically active compounds comprising a primary transcriptional unit and one or more secondary transcriptional units, a primary transcriptional unit encoding promoter, synthetic intron, selectable marker gene and polyadenylation signal or transcriptional terminator and a second transcriptional unit encoding promoter and polypeptide of interest surrounded by insulator sequences and placed in the intron of primary transcriptional unit. The synthetic intron disclosed is positioned at the 5′ end of the coding sequence and the synthetic intron capable for accommodating secondary transcriptional unit with base pairs ranging from 500 to 6000 and more.

### TECHNICAL FIELD

The present subject matter generally relates to the field of expression of biologically active proteins in host cells. More particularly the present subject matter relates to construction of an expression cassette with the protein of interest and methods for expressing the protein in host cells.

### BACKGROUND

In the recent years, the discovery of methods for introducing DNA into living host cells in a functional form has provided the key to understand many fundamental biological processes. These methods are used to produce many important proteins and other molecules in commercially useful quantities.

Generally, the above disclosed methods includes several common problems that may limit the efficiency with which a gene encoding a desired protein can be introduced into and expressed in a host cell. A problem is distinguishing between the cells that contain the GOI (gene of interest) and the cells that have survived the transfer procedures but do not contain the GOI. Another problem is identifying and isolating the cells that contain the gene and that are expressing high levels of the protein encoded by the gene.

Further, identification and over-expression of novel genes associated with human disease is an important step towards developing new therapeutic drugs. The cloning of cDNA is carried to produce protein over-expression of cells and these cells are deposited in a depository library. Thus in order to identify a new gene using this approach, the gene must be expressed in the cells at sufficient levels to be adequately represented in the depository library. This is problematic because many genes are expressed only in very low quantities, in a rare population of cells or during short developmental periods.

Furthermore, because of the large size of some mRNAs it is difficult or impossible to produce full length cDNA molecules capable of expressing the biologically active protein. Lack of full-length cDNA molecules has also been observed for small mRNAs and is thought to be related to sequences in the message that are mammalian expression systems to produce by reverse transcription or that are unstable during propagation in bacteria. As a result, even the most complete cDNA depository libraries express only a fraction of the entire set of possible genes.

In the light of the aforementioned discussion, there exists a need for new methods and new full length constructs for constant expression of extremely valuable biologically active proteins in mammalian host cells.

### BRIEF SUMMARY

The following presents a simplified summary of the disclosure in order to provide a basic understanding to the reader. This summary is not an extensive overview of the disclosure and it does not identify key/critical elements of the disclosure or delineate the scope of the invention.

Exemplary objective of the subject matter is to provide a DNA molecule comprising a primary transcriptional unit coding for promoter, synthetic intron, a selectable marker polypeptide functional in eukaryotic host cells, a polyadenylation signal or transcriptional terminator. The synthetic intron of the primary transcriptional unit contains second transcriptional unit coding for promoter and polypeptide of interest.

Another exemplary objective of the present subject matter is to provide a DNA molecule comprising a primary transcriptional unit coding for Promoter, Synthetic intron, a selectable marker polypeptide functional in eukaryotic host cells, a polyadenylation signal or transcriptional terminator. The synthetic intron of the primary transcriptional unit containing two transcriptional units encoding Promoter, amplifiable gene or a fluorescent reporter protein and promoter, polypeptide of interest.

Another exemplary objective of the present disclosure is, the selectable marker protein provides resistance against lethal and/or growth-inhibitory effects of a selection agent, such as an antibiotic.

Another exemplary objective of the present disclosure is to develop a regulatable expression of selectable marker protein using inducible promoter.

Another exemplary objective of the present disclosure is to develop a coding sequence of the polypeptide of interest comprising an optimal translation start sequence.

Another exemplary objective of the present disclosure is to develop a synthetic intron which can accommodate all the necessary sequences for better expression and capable of splicing.

Another exemplary objective of the present disclosure is to develop a synthetic intron which can be as long as 500 base pairs to 6000 base pairs and more.

In certain embodiments, the polypeptide of interest is a part of a multimeric protein, for example a heavy or light chain of an immunoglobulin. The invention also provides host cells comprising DNA molecules according to the invention.

### BRIEF DESCRIPTION OF THE DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a schematic representation of the use of multiple promoters in tandem to drive the expression of selectable marker gene and the polypeptide of interest.

FIG. 2 is a schematic representation of the use of multiple promoters in tandem to drive the expression of selectable marker gene, amplifiable gene/reporter protein gene and polypeptide of interest.

FIGS. 3A-3G are figures showing plasmids carrying varying lengths of synthetic intron.

FIGS. 4A-4C are figures showing construction of pUB-CE-100-N Plasmid.

FIGS. 5A-5C are figures showing construction of pUB-CE-100-N-GFP. pUB-CE-100-N-GFP was constructed by ligating BgIII and NotI fragment (3555bp) of pUB-CE-100-N with BgIII and Nod fragment (1540bp) of pUB-GFR

FIG. 6 is a figure showing comparison of expression between different vectors containing synthetic intron varying in size from 500 to 6000 base pairs.

FIG. 7 is a figure showing transient expression assay to test the functionality of pUB-CE-100-N-GFP plasmid.

FIG. 8 is a figure showing comparison of GFP expression between CHOK1 stable pools developed using pUB-GFP and pUB-CE-100-N-GFP

FIG. 9 is a figure showing comparison of GFP expression between GFP expressing stable pool and clone developed using pUB-CE-100-N-GFP

FIG. 10 is a figure showing construction of pUB-CE-100-N-Ab-Lc and pUB-CE-100-H-Ab-Hc Plasmids: pUB-CE-100-N-Ab-Lc was constructed by ligating BglII and NotI fragment (3555bp) of pUB-CE-100-N with BgIII and NotI fragment of (1510bp) of pUB-Ab-Lc plasmid.pUB-CE-100-H-Ab-Hc was constructed ligating BgIII and NotI fragment (3837bp) of pUB-CE-100-H with BgIII and NotI fragments (2224bp) of pUB-Ab-Hc plasmid.

FIG. 11 is a graph showing fed-batch study of mAb producing clone developed using pUB-CE-100-N-Ab-Lc and pUB-CE-100-H-Ab-Hc.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

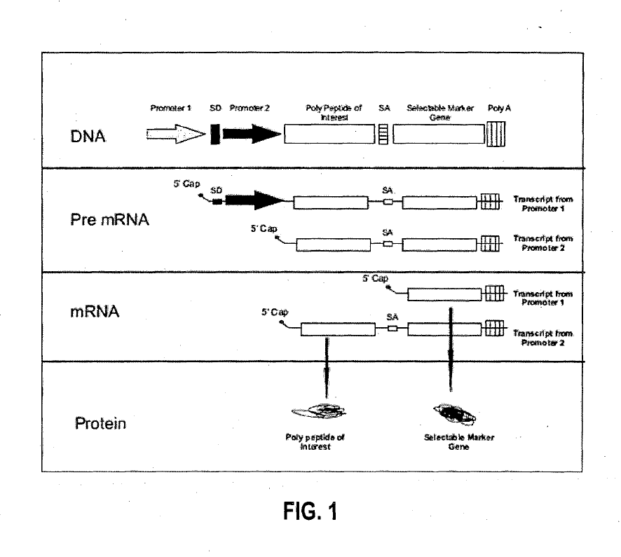

FIG. 1 is a schematic representation of the use of multiple promoters in tandem to drive the expression of selectable marker gene and the polypeptide of interest in one transcriptional unit. Promoter-1-Synthetic intron-neomycin-Polyadenylation signal being the primary transcriptional unit and Promoter-2-Polypeptide of interest being the second transcriptional unit and is part of the synthetic intron.

According to an exemplary aspect of the present disclosure, transcription from promoter-1 results in expression of selectable marker gene due to splicing of synthetic intron formed by Splice donor (SD) and Splice acceptor (SA).

In accordance with a non limiting exemplary aspect of the present disclosure, transcription from promoter 2 results in the expression of polypeptide of interest as eukaryotic transcriptions are 5′ cap dependent. Polypeptide of interest will be expressed but not the selectable marker gene.

According to an exemplary aspect of the present disclosure, Promoter I can be inducible promoter to regulate the expression of selectable marker gene there by allowing better selection. Promoter 2 can be a constitutive promoter which can result in high expression of polypeptide of interest.

In accordance with a non limiting exemplary aspect of the present disclosure, cloning of Promoter 2 and Polypeptide of interest in the intron of primary transcriptional unit will generate 100% expressing stable pool for polypeptide of interest following selection. There by increasing the chances of isolation of high expressing cell line.

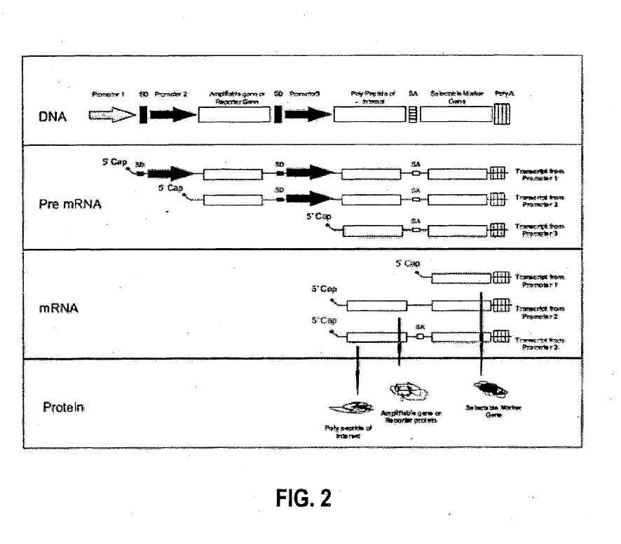

FIG. 2 is a schematic representation of the use of multiple promoters in tandem to drive the expression of selectable marker gene, amplifiable gene or reporter protein gene and Polypeptide of interest in one transcriptional unit. Promoter-1-Synthetic intron-Selectable marker gene-Polyadenylation signal is the primary transcriptional unit and Promoter-2-Amplifiable gene and Promoter 3-Polypeptide of interest are the secondary transcriptional unit cloned in the synthetic intron of primary transcriptional units in tandem. In accordance with a non limiting exemplary aspect of the present disclosure, transcription from promoter-1 results in expression of selectable marker gene due to splicing of synthetic intron formed by Splice donor-1(SD-1) and Splice acceptor (SA).

According to an exemplary aspect of the present disclosure, transcription from promoter 2 results in the expression of amplifiable gene or reporter gene. Eukaryotic transcriptions are 5′ cap dependent. Amplifiable gene or reporter protein will be expressed but not the selectable marker gene.

In accordance with a non limiting exemplary aspect of the present disclosure, transcription from promoter 3 results in the expression of polypeptide of interest as eukaryotic transcriptions are 5′ cap dependent. Polypeptide of interest will be expressed but not the selectable marker gene.

According to an exemplary aspect of the present disclosure, promoter 1 can be inducible promoter to regulate the expression of selectable marker gene there by allowing better selection and promoter 2 can be inducible promoter to regulate the expression of amplifiable gene or reporter protein gene which can be switched on as and when required. The promoter 3 can be a constitutive promoter which can result in high expression of polypeptide of interest.

In accordance with a non limiting exemplary aspect of the present disclosure, cloning of Promoter 2-amplifiable gene or reporter protein and Promoter 3-Polypeptide of interest in the intron of primary transcriptional unit will generate 100% expressing stable pool for amplifiable gene or reporter protein and polypeptide of interest following selection. The amplifiable gene or reporter protein will help better amplification or selection for high expressing cell line and all the cells expressing amplifiable gene will also express high amount of polypeptide of interest there by facilitating isolation of high expressing cell line.

In accordance with a non limiting exemplary aspect of the present disclosure the DNA molecules comprise of a sequence encoding a functional selectable marker polypeptide, characterized in that such DNA molecules comprise a mutation that decreases the translation initiation efficiency of the functional selectable marker polypeptide in a eukaryotic host cell. Preferably, such a DNA molecule comprises a GTG or a TTG start codon followed by an otherwise functional selectable marker coding sequence.

According to an exemplary aspect of the present disclosure, a method for generating host cells expressing a polypeptide of interest is disclosed. The method comprises of introducing an expression cassette to a plurality of precursor host cells, culturing the cells under conditions selecting for expression of the selectable marker polypeptide and selecting one or more host cell producing the polypeptide of interest.

In accordance with a non limiting exemplary aspect of the present disclosure methods for producing a polypeptide of interest is disclosed. The methods comprises of culturing a host cell and the host cell comprising an expression cassette and expressing the polypeptide of interest from the expression cassette. In preferred embodiments thereof, the polypeptide of interest is further isolated from the host cells and/or from the host cell culture medium.

According to an exemplary aspect of the present disclosure, the expression cassettes further comprises of at least one chromatin control element chosen from the group consisting of a matrix or scaffold attachment region (MAR/S AR), an insulator sequence, a ubiquitous chromatin opener element (UCOE) and an anti-repressor sequence. The expression cassettes are further positioned upstream of the promoter driving expression of the polypeptide of interest and downstream of the polypeptide of interest in the synthetic intron.

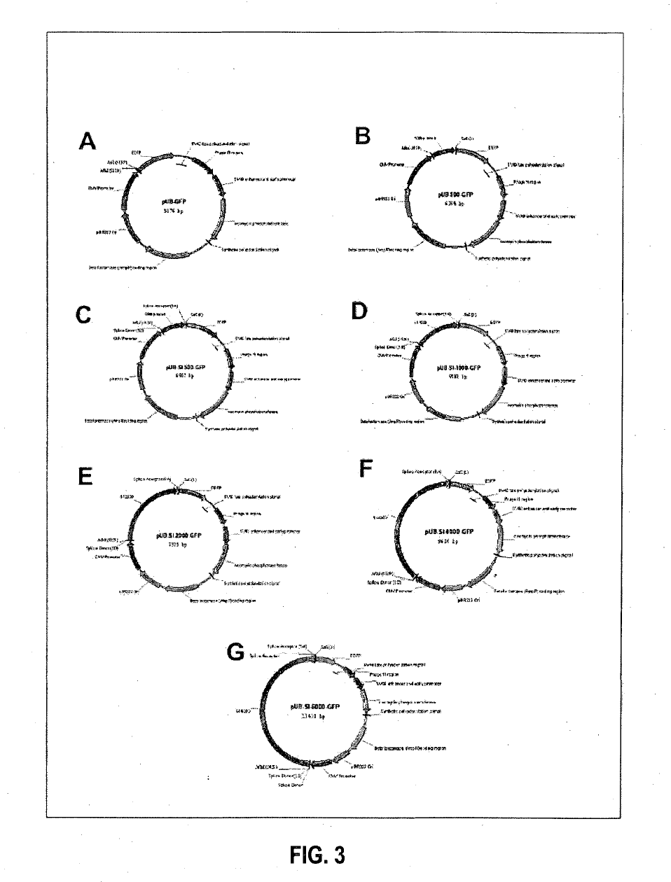

Referring to FIGS. 3A-3G are figures showing plasmids carrying varying lengths of synthetic intron. Plasmids used in this project were purified using different techniques for different applications. Plasmids used for cloning were routinely isolated by the alkaline lysis method or by using UB-Plasmid Mini Kit (Usha Biotech Ltd, Hyderabad). However, for transfection of mammalian cells, plasmids were isolated using the UB-Plasmid Midi Kit (Usha Biotech Ltd, Hyderabad)

### Desalting of DNA

Digested plasmid DNA was routinely purified using UB-Desalting Kit.

### Restriction Digestion

5-10 ug of DNA was routinely digested with 1-5 units (U) of enzyme in the appropriate reaction conditions described by the manufacturer. The reaction was usually carried out in 20 ul reaction volume at the recommended temperature for 1-2 h. the DNA fragments were visualized in a UV transilluminator and gel documentation system (SynGene, Cambridge, UK) following electrophoresis on 0.8-1% agarose gel. Commercially available DNA size marker (1 kb and 100 by DNA ladders) were run along with the digested samples to compare and estimate the size of the restriction fragment.

### Agarose Gel Electrophoresis

Plasmid DNA separation was routinely performed on 0.8 to 1% agarose gel in 1×Tris Acetic acid: EDTA (TAE) electroporation buffer pH 8.3 (2 mM Tris-Acetate/0.05 M EDTA). Agarose gels were cast in 1×TAE buffer containing 0.5 μg/ml of ethidium bromide. DNA samples were mixed with ⅙^{th} volume of 6×loading dye (NBE, Beverly, Mass.) and subjected to electrophoresis under controlled voltage of 5 V/cm. Appropriate DNA size markers (1 Kb or 100 bp DNA ladder) were run alongside the samples to estimate the size and concentration of the DNA fragments. The DNA was visualized in an UV transilluminator and gel documentation system (SynGene, Cambridge, UK).

### Transient Transfection

### Lipofection of CHO-K1

Lipofections were carried out using the Lipofectamine 2000 reagent (Invitrogen) according to the manufacturer's directions. Briefly, 5 ug of DNA in 250 ul of OptiMEM media and 15 ul of Lipofectamine 2000 in 250u1 of OptiMEM media were prepared at room temperature and incubated for 5 min. The DNA and lipofectamine2000 were combined and incubated together for a further 20minutes before adding to cells at 70 to 80% confluence in 6 well plate. Cells were analyzed using FACS (BD) after 48 hours.

### Stable Transfection

### Electroporation

Electroporation was routinely used for the development of stable cell line. CHO-K1 cells at an exponential growth phase (70-80% confluence) were detached with EDTA/PBS, washed once with 1×PBS and resuspended at 5×10⁶ cells/ml in electroporation buffer. 200 ul of resuspended cells were aliquoted into an electroporation cuvette (2 mm) (sigma) and 2 μg of linear DNA was added to the cuvettes, with the exception of a negative control where equal volume of 1×PBS was added. Cells were pulsed at 550 V, 40 μsec, 1 pulse using Multiporator (eppendorf). After pulsing cells were incubated at 37° C. for 10 min before transferring the cells to 5 ml of growth media. Cells were centrifuged at 800 rpm for 10 min and then resuspended in 12 ml of growth medium and flasked in T75 (BD, India). Twenty four hours post transfection cells were selected with 1 mg/ml G418.

### Flow Cytometry Analysis

All data presented were gathered on BD™ LSR II Flow cytometer, tuned to Blue Laser (488 nm Excitation Wave Length). Data was analyzed on High Performance BD FACSDiva Software. Forward and Side-Scatter light gating were used to identify viable population whilst doublets were excluded using forward angle and pulse-width scatter gating. Analysis was maintained at an event rate not exceeding 600 cells per second and a total of 25,000 events were acquired per sample.

### Single Cell Cloning By Limiting Dilution Method

Stable clones were routinely isolated by limiting dilution method. On the day of plating cell count was performed and cell were diluted to 5 cells/ml in growth media and plating at 200 ul/well. Plates were then incubated in 37° C. incubator for 15 days. Well with single clones were marked by observing under microscope for further use.

### Fed-Batch Study for Monoclonal Antibody Productivity

Fed-Batch Study was performed in 250 ml shake flask. On day 0 cells were centrifuged and resuspended at 5×10⁵ cells/ml in Power CHO2 Media. Flask was incubated in 37° C. incubator with shaking. 1 ml of culture was collected very 24 hr to determine cell count and antibody productivity.

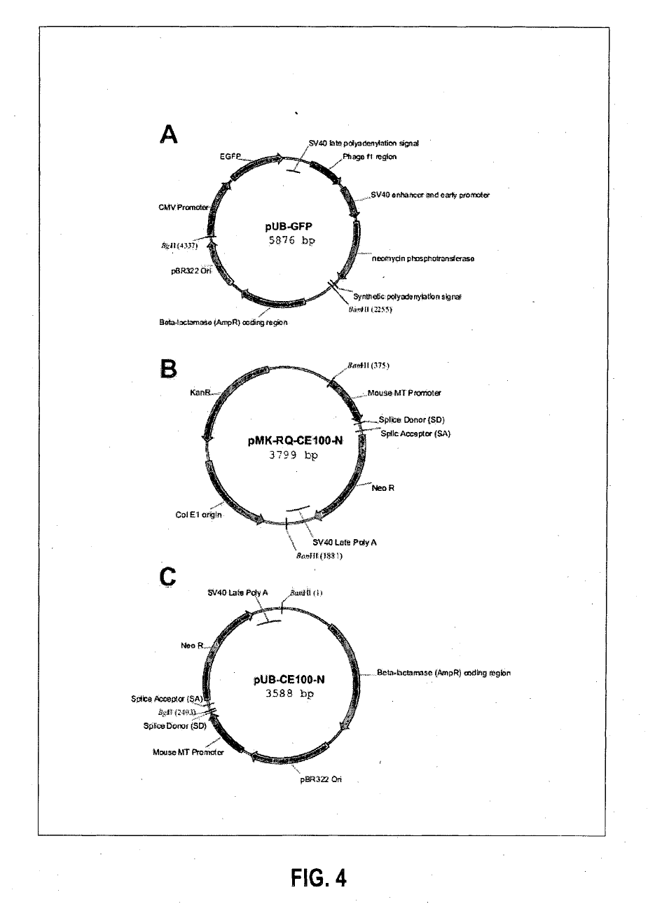

Referring to FIG. 4A-4C are figures showing construction of pUB-CE-100-N Plasmid. pUB-CE-100-N was constructed by ligating BamHI and BgIII fragment (2084 bp) of pUB-GFP plasmid with BamHI fragment (1506 bp) of pMK-RQ-CE100-N plasmid grown in JM109.

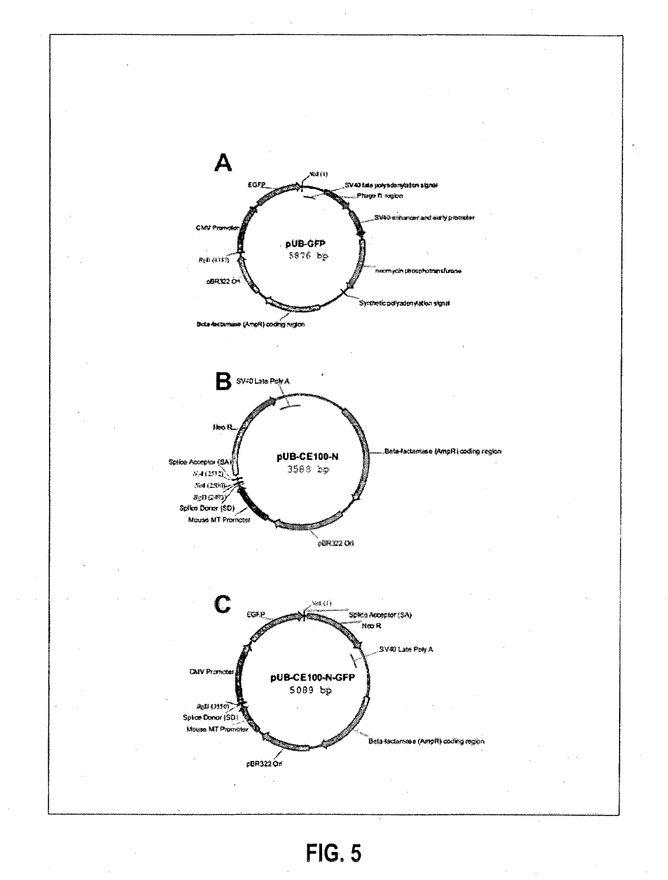

Referring to FIG. 5A-5C are figures showing construction of pUB-CE-100-N-GFP. pUB-CE-100-N-GFP was constructed by ligating BgIII and NotI fragment(3549 bp) of pUB-CE-100-N with BgIII and NotI fragment (1540 bp) of pUB-GFP.

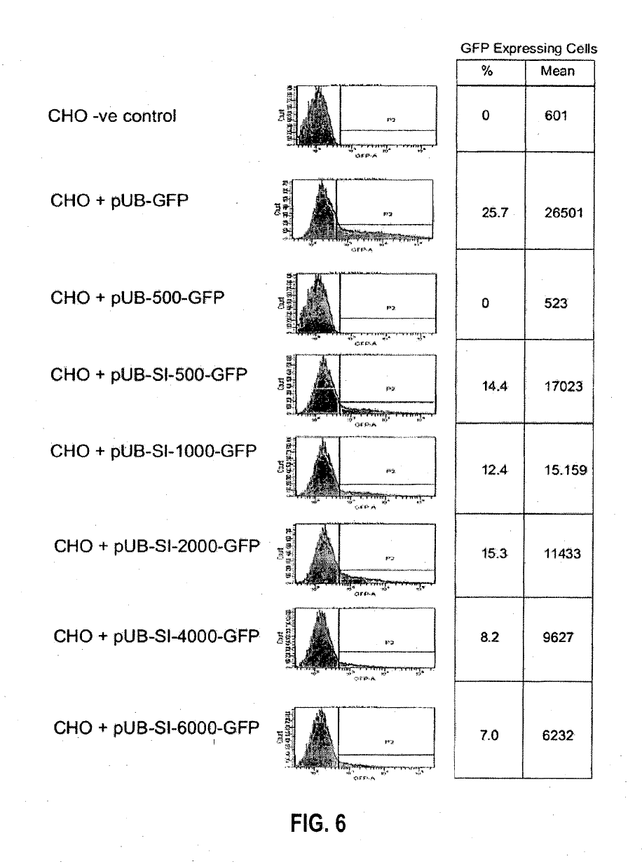

Referring to FIG. 6 is a graph showing comparison of expression between different vectors containing synthetic intron varying in size from 500 to 6000 base pairs.

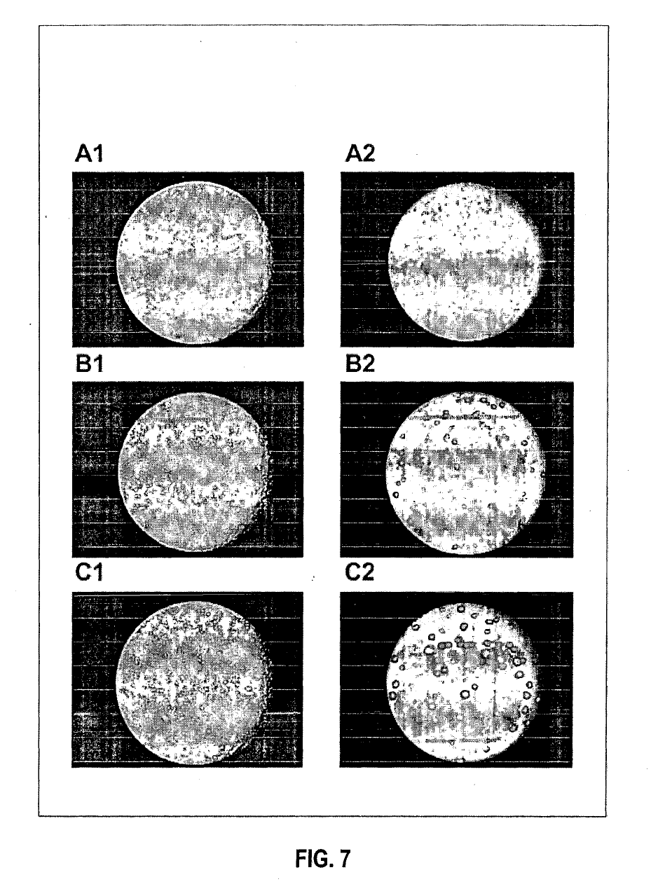

Referring to FIG. 7 is a figure showing transient expression assay to test the functionality of pUB-CE-100-N-GFP plasmid.

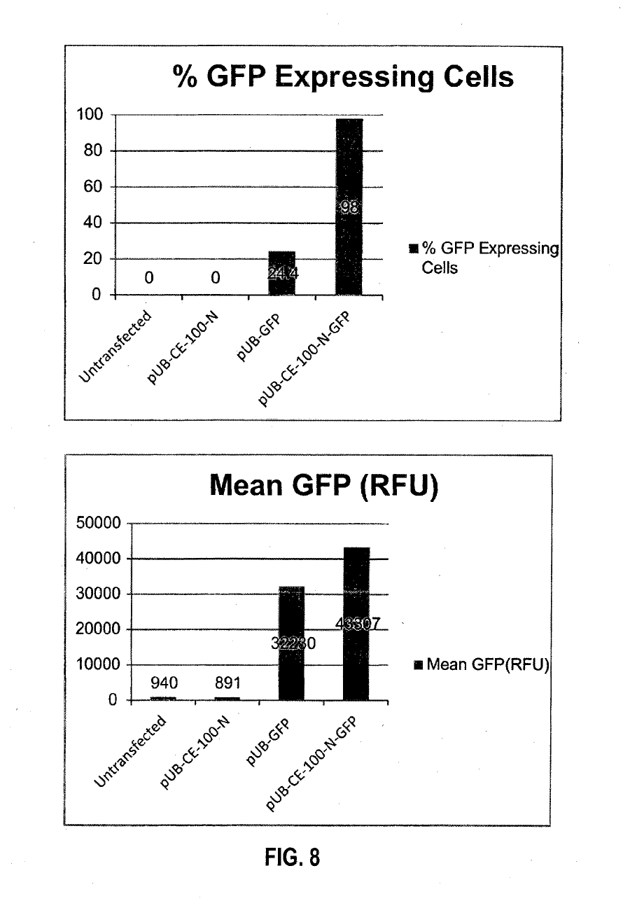

Referring to FIG. 8A and 8B are graphs showing comparison of GFP expression between CHOK1 cells stably transfected with pUB-GFP and pUB-CE-100-N-GFP.

TABLE 1

| Oligo Name | Oligo Sequence (5′ to 3′) |
|---|---|
| 500-F | atgagggggatgctgcccct |
| 500-R | caggccggggtgatgaggta |
| SI-500-F | acgcgtgggtgagtctctagagatgagggggatgctgccc |
| | ct |
| SI-500-R | gtcgacgagctgtaggaaaaagaagaaggcatgcaggccg |
| | gggtgatgaggta |
| SI-1000-R | gtcgacgagctgtaggaaaaagaagaaggcatgggtggac |
| | ccggccccccctg |
| SI-2000-R | gtcgacgagctgtaggaaaaagaagaaggcatgaagttga |
| | tggtcttggccgc |
| SI-4000-R | gtcgacgagctgtaggaaaaagaagaaggcatggatctgg |
| | gtccaacttacc |
| SI-6000-R | gtcgacgagctgtaggaaaaagaagaaggcatgttaacat |
| | gaccttttacatgg |

Plasmids with varying lengths of synthetic intron's (pUB-SI-500-GFP, pUB-SI-1000-GFP, pUB-SI-2000-GFP, pUB-SI-4000-GFP, pUB-SI-6000-GFP) were constructed by PCR amplification using Forward primer carrying Sequence for Splice Donor and Reverse primer carrying sequence for Splice Acceptor (Table-1). PCR amplified fragment were cloned into pGEM-T easy vector and then sub cloned into pUB-GFP plasmid using SaII and mluI restriction ends. For the construction of pUB-500-GFP PCR amplification was carried out with Splice Donor and Acceptor sites on the oligos.

### EXAMPLE 1

### Analysis of Intron Function with Respect To Size

A series of expression vectors (pUB-SI-500-GFP, pUB-SI-1000-GFP, pUB-SI-2000-GFP , pUB-SI-4000-GFP , pUB-SI-6000-GFP) as shown in HG 3A to 3G were constructed to demonstrate the effect of size of intron on splicing. Synthetic introns (SI-500, SI-1000, SI-2000, SI-4000, SI-6000) were constructed by. PCR amplification of Taq DNA coding sequences with Forward and Reverse Primers having minimal splice donor and minimal splice acceptor sequences of Beta-Globin Large Intron Sequence. A plasmid (pUB-500-GFP) with 500 by fragment with out splice donor and splice acceptor sequence was also constructed to have a control for expression in the absence of splicing.

The expression vectors pUB-GFP, pUB-500-GFP, pUB-SI-500-GFP, pUB-SI-1000-GFP, pUB-SI-2000-GFP , pUB-SI-4000-GFP , pUB-SI-6000-GFP (FIG. 3) were transfected in to CHOK1 cells using Lipofectamine 2000° . Forty eight hours post transfection cell were analyzed for GFP expression using BD™ LSR II Flow cytometer. The mean GFP expression and % GFP expressing cells were compared and were as shown in FIG. 6.

All the Synthetic Intron containing plasmids showed GFP expression following transient transfection. pUB-500-GFP didn't show any GFP expressing cells indicating that the expression from synthetic intron containing plasmids is due to splicing. However, the mean GFP expression and % GFP expressing cells decreased with increase in the size of the synthetic intron. The decrease in the mean GFP and % GFP expressing cells with increase in size of synthetic intron could be due to the size of the plasmid. Size of plasmid is known to affect transient transfection efficiency.

The above experiment had demonstrated that the Minimal Synthetic Donor Sequence and Minimal Synthetic Accepter Sequence can accommodate upto 6000 by sequence.

### EXAMPLE 2

### Transient Expression Assay to Test Expression of GFP from Secondary Transcriptional Unit Cloned in the Intron of Primary Transcriptional Unit

To test functionality of secondary transcriptional unit, CMV-GFP was cloned in the 5′ intron of primary transcriptional unit which encodes for Neomycin Resistance Gene (FIG. 5C). To test the expression of GFP, pUB-CE-100-N-GFP was transfected in to CHOK1 cells using Lipofectamine method. Forty eight hours post transfection GFP expressing cells were analyzed by Fluorescent microscopy. pUB-GFP (positive control) (FIG. 7C1 and C2) and pUB-CE-100-N (negative control) (FIG. 7A1 and A2 were used as controls in the experiment. Presence of GFP expression in pUB-CE-100-N-GFP transfected cells as shown in FIG. 7B1 and B2 indicated that the positioning of the secondary transcriptional unit in the intron of primary transcriptional unit didn't affect the expression of GFR

### EXAMPLE 3

### Analysis of Functionality of Primary and Secondary Transcriptional Unit'S Following Stable Transfection

Primary transcriptional unit is often antibiotic selectable marker gene which was under the control of inducible metallothionein promoter and Secondary transcriptional unit is often Polypeptide of Interest which is under the control of a constitutive CMV promoter. The use of inducible promoter will help to switch off expression of neomycin resistance gene after selection. To test the functionality of both the primary and secondary transcriptional unit's pUB-CE-100-N-GFP was transfected in to CHOK1 cells using electroporation. Expression of Neomycin resistance gene was induced with 25 nm ZnSo₄ immediately after transfection. Twenty four hours post transfection cells were selected with 1 mg/ml G418. Fifteen days post transfection and selection, G418 resistant cells were analyzed for GFP expression using BD^{TM} LSR II Flow cytometer. pUB-GFP (control for expression of neomycin and GFP) and pUB-CE-100-N (control for expression of neomycin resistance gene) were also transfected in to CHOK1 cells and selected with 1 mg/ml G418.

All the plasmids (pUB-GFR pUB-CE-100-N, pUB-CE-100-N-GFP) gave rise to G418 resistant colonies following selection indicating the presence of expression of neomycin resistance gene in all the transfectants. However, the efficiency of stable integration was found to be more in pUB-GFP compared to pUB-CE-100-N and pUB-CE-100-N was found to be more compared to pUB-CE-100-N-GFP (Table 2). The decrease in the number of G418 resistant colonies in pUB-CE-100-N-CMV-GFP could be due to the positioning of secondary transcriptional unit (CMV-GFP) in intron of primary transcriptional unit, (neomycin resistance gene). The possible reasons could be read through transcription and promoter occlusion.

TABLE 2
Number of G418 Resistant Colonies Found 15 Days Post
Transfection and Selection

| | No. of G418 Resistant |
|---|---|
| | Colonies following |
| Sample | Selection |
| CHOK1 Transfected with No Plasmid | 0 |
| CHOK1 Transfected with pUB-GFP | 1.42 |
| CHOK1 Transfected with pUB-CE-100-N | 67 |
| CHOK1 Transfected with pUB-CE-100-N-GFP | 23 |

Fifteen days post transfection and selection stable pools were analyzed for GFP expression using BD™ LSR II Flow cytometer. Mean GFP and % expressing cells were compared between pUB-GFP, pUB-CE-100-N and pUB-CE-100-N-GFP. pUB-CE-100-N-GFP showed 0.3 fold higher mean GFP expression and 5 fold high %GFP expressing cells compared to pUB-GFP (FIG. 8). The possibility of auto fluorescence was ruled out with the lack of GFP expression in untransfected samples and pUB-CE-100-N transfected samples. The presence of high mean GFP expression and % GFP expression cells in pUB-CE-100-N-GFP could be due to better selection.

The above experimentation had demonstrated that position of secondary transcriptional unit (GFP) in the intron of primary transcriptional unit (neomycin resistance gene) had affected the expression of neomycin resistance gene but not the GFP gene. The design further helped in the generation high expressing stable pool compared to normal plasmid. The high expressing pool will further help in the quick isolation of high expressing cell line.

### EXAMPLE 4

### Comparison of GFP Expressing Stable Pool and Clone Generated Using pUB-CE-100-N-GFP

pUB-CE-100-N-GFP with its unique design and stringent selection conditions results in high expressing stable pool that resembles that of clone. To compare Mean GFP expression and % expressing population between clone and pools stable transfection was repeated as in Example 3. Twenty four hours post transfection cells were selected at 1 mg/ml in G418 in T75 flask to generate stable pool and 96 well plate to generate stable clones. Fifteen days post selection, G418 resistant pool and clones were analysed for GFP expression using BD™ LSR II Flow Cytometer.

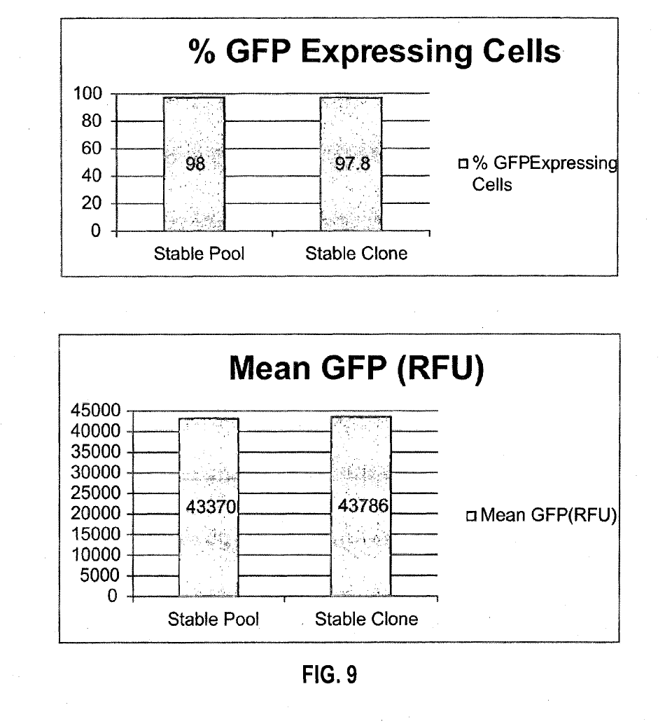

pUB-CE-100-N-GFP stable pools resemble that of clone with respect to mean GFP expression and % GFP expression population (FIG. 9). In order to test the stability of pool, the pool was cultures for 30 days in the absence of selection. Stable pool were found .to be quiet stable for more than 30 days with respect to % GFP expression population. However, there is a slight drop in mean GFP expression.

High % GFP expression cell, High mean GFP and High stability makes pUB-CE-100-N-GFP stable pools ideal for scale up to bioreactor early in drug development for pre-clinical material generation.

### EXAMPLE 5

### Fed Batch Study of Antibody Producing Clone Generated Using pUB-CE-100-N-Ab-Lc and pUB-CE-100-H-Ab-Hc Plasmids

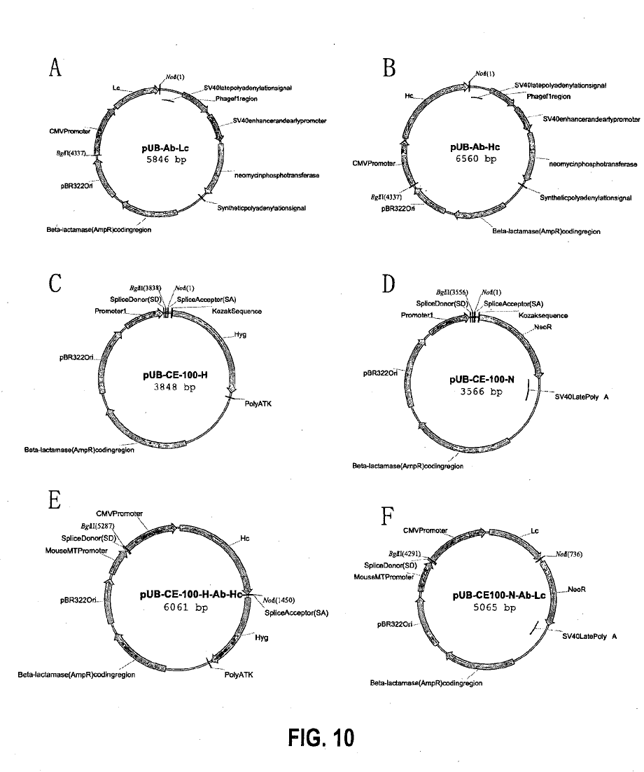

Antibody productivity by the vector system of the invention was tested by co-transfection of light chain and heavy chain plasmids wherein the light chain was placed in the intron of neomycin resistance gene in pUB-CE-100-N-Ab-Lc (FIG. 10) and heavy chain plasmid was placed in the intron of hygromycin resistance gene in pUB-CE-100-H-Ab-Hc (FIG. 10). To test the efficiency of the expression vector of the invention pUB-CE-100-N-Ab-Lc and pUB-CE-100-H-Ab-Hc were co-transfected into CHOK1 cells using electroporation. Expression of Neomycin Resistance Gene and Hygromycin Resistance Gene were induced with 25 nm ZnSo4 immediately after transfection. Twenty four hours post transfection cells were selected with 1 mg/ml G418 and 200 ug/ml Hygromycin. Fifteen days post transfection and selection G418 and Hygromycin resistant cells were analyzed for antibody productivity and cell were plated in 96 well plate for isolation of clones. 15-20 days post plating clones were analyzed for productivity and one best clone was picked for Fed-Batch study.

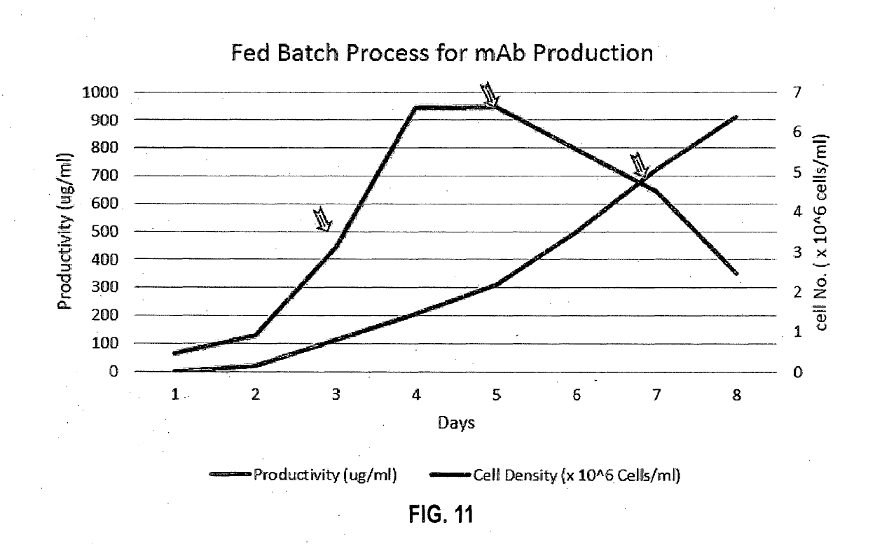

Monoclonal antibody productivity was analyzed in Fed-Batch mode which is often the method of choice for antibody production. Fed batch study was carried out in 250 ml shake flaks. Antibody producing clone was seeded at 5×10⁵ cells/ml in 70 ml of Power CHO2 media. Culture was fed with Cell Boost 5 at 5% volume on day 3^{rd}, 5^{th}, 7^{th}and 7^{th}. Samples were collected every 24 hr to determine cell count (Haemocytomer) and antibody productivity (ELISA). Cell density and antibody productivity were plotted and were shown in FIG. 11. From the data it was clear that antibody producing clone had displayed a maximum cell density of 6.64×10⁶ cells/ml and a maximum productivity of 911 mg/L.

Also, those skilled in the art can appreciate from the foregoing description that the present invention can be implemented in the variety of forms. Therefore, while the embodiments of this invention have been described in connection with particular examples thereof, the true scope of the embodiments of the invention should not be so limited since other modifications will be apparent to the skilled practitioner upon a study of the drawings and following claims.

1. An expression cassette for production of biologically active compounds, comprising:
a primary transcriptional unit comprising at least one of a secondary transcriptional unit;
wherein the primary transcriptional unit further comprises sequences for a promoter 1, a synthetic intron, a selectable marker gene and at least one of a polyadenylation signal and a transcriptional terminator;
wherein the synthetic intron is positioned at the 5′ end of the coding sequence of the selectable' marker gene.

2. The expression cassette according to claim 1, wherein the selectable marker gene is at least one of a neomycin resistance gene and a hygromycin resistance gene.

3. The expression cassette according to claim 1, wherein the secondary transcriptional unit comprises of base pairs ranging from about 500 to about 6000.

4. The expression cassette according to claim 1, wherein the promoter 1 is an inducible promoter.

5. The expression cassette according to claim 1, wherein the secondary transcriptional unit comprises sequences for a promoter 2 and a polypeptide of interest surrounded by an insulator sequence and placed in the synthetic intron of the primary transcriptional unit.

6. The expression cassette according to claim 5, wherein the polypeptide of interest is at least one of a light chain of a monoclonal antibody and a heavy chain of a monoclonal antibody.

7. The expression cassette according to claim 5, wherein the promoter 1 and the promoter 2 are in tandem in head to tail orientation.

8. The expression cassette according to claim 5, wherein the expression cassette is cloned in a vector capable of replication in at least one of a prokaryotic host and an eukaryotic host.

9. The expression cassette according to claim 1, wherein the primary transcriptional unit comprises of a first secondary transcriptional unit and a second secondary transcriptional unit placed in tandem;
wherein the first secondary transcriptional unit comprises sequences for a promoter 2 and at least one of an amplifier gene and a reporter gene;
wherein the second secondary transcriptional unit comprises sequences for a promoter 3 and a polypeptide of interest.

10. The expression cassette according to claim 9, wherein the polypeptide of interest is at least one of a light chain of a monoclonal antibody and a heavy chain of a monoclonal antibody.

11. The expression cassette according to claim 9, wherein the promoter 1, the promoter 2 and the promoter 3 are in tandem in head to tail orientation.

12. The expression cassette according to claim 9, wherein the expression cassette is cloned in a vector capable of replication in at least one of a prokaryotic host and an eukaryotic host.

13. A method of expressing a protein of interest in eukaryotic cells comprising:
constructing an expression cassette comprising a primary transcriptional unit comprising at least one of a secondary transcriptional unit; wherein the primary transcriptional unit further comprises sequences for a promoter 1, a synthetic intron, a selectable marker gene and at least one of a polyadenylation signal and a transcriptional terminator; wherein the secondary transcriptional unit encodes for at least one of a polypeptide of interest and an amplifier gene;
cloning the expression cassette in a vector;
transfecting the vector into an eukaryotic cell;
growing the eukaryotic cells in a selection media for selection of stable cells;
growing the eukaryotic cells in a nutrient medium; and
harvesting the protein of interest after its expression.
